(19) United States
(12) Patent Application Publication
(10) Pub. No.: US 2024/0347140 A1
(43) Pub. Date: Oct. 17, 2024

(54) METHOD FOR DEVISING OR DESIGNING A TECHNICAL OR CHEMICAL PROCESS, AND GUI WITH SAID FUNCTION

(71) Applicant: FRAUNHOFER-GESELLSCHAFT ZUR FOERDERUNG DER ANGEWANDTEN FORSCHUNG E.V., Muenchen (DE)

(72) Inventors: Michael BORTZ, Kaiserslautern (DE); Karl-Heinz KUEFER, Weilerbach (DE); Philipp SUESS, Budenheim (DE)

(21) Appl. No.: 18/558,517

(22) PCT Filed: May 3, 2022
(86) PCT No.: PCT/IB22/54077

§ 371 Date: Jun. 24, 2024

(30) Foreign Application Priority Data
May 3, 2021 (DE) 10 2021 111 421.7

(51) Int. Cl. G16C 20/10; G06F 3/0481; G06F 3/0484; G16C 20/80

(52) U.S. Cl. CPC G16C 20/10; G06F 3/0481; G06F 3/0484; G16C 20/80

(57) ABSTRACT

The aim of the invention is to provide a procedure which enables or improves multi-criteria optimization of a chemical or procedural process. Interaction between a user and an optimization tool is to be improved as well. This is achieved by interactive navigation on a display device (10). At least a first and a second configuration (A, B, . . . Z) are available for selection, and at least a first Pareto frontier (101, . . . ) is formed for the first configuration (A) and at least a second Pareto frontier (201, . . . ) for the second configuration (B). Based on the first Pareto frontier (101, . . . ) for the first configuration (A) and the second Pareto frontier (201, . . . ) for the second configuration (B), a resulting Pareto frontier (801) is or will be formed. A first operating aid (21) and a second operating aid (22) are shown on the display device (10) in an operating area (2), with the first operating aid (21) representing a first criterion (c1) and the second operating aid (22) representing a second criterion (c2). A position or setting of a selector (21a) of the first operating aid (21) represents a value of the first criterion (c1), and a position or setting of a selector (22a) of the second operating aid (22) represents a value of the second criterion (c2). Moving or operating one of the selectors (21a, 22a) of the respective operating aid (21, 22) changes the value of the respective criterion (c1, c2). Based on the position or setting of the selector (21a, 22a), a point (zᵢ) is determined and/or shown on the resulting Pareto frontier (801). A system, a computer program product for designing or devising the procedural or chemical process is also proposed.

The invention involves a procedure for designing or devising a procedural or chemical process, specifically by using multi-criteria optimization. The invention also involves an interactive graphical user interface (GUI) for multi-criteria optimization of a process.

Conflicting requirements often need to be reconciled when planning chemical or process engineering processes. In product development, for example, a composition with both a high degree of hardness and high elasticity may be required. Hard components are often relatively inelastic, and elastic components are often relatively soft. The proportions of a hard component and an elastic component can be varied in order to provide a composition with acceptable hardness and elasticity, whereby the improvement of one property (e.g. hardness of the composition) generally causes a deterioration of another property (e.g. elasticity of the composition). A compromise of the required properties can be achieved through multi-criteria optimization.

In the field of chemical synthesis, for example, there may be a trade-off between the degree of purity and the availability of a reactant. Reactants with a relatively high degree of purity may not be available for several months, while similar reactants with a lower degree of purity may be available immediately. Several reactants of the same type may be available, with the reactants possibly differing in their degree of purity and the readiness of their availability. Multi-criteria optimization allows the properties of the reactants to be weighed against one another.

A material separation process (e.g., rectification) with a high degree of purity of the separated components and low energy consumption may also be required. Frequently, a high degree of purity of a component separated from a mixture can only be attained by using a high amount of energy.

Multi-criteria optimization is not trivial, especially if there are a large number of possibilities—for example, if there are a large number of possible components in the product development process referred to above. The planning of such a process can overwhelm a user who is designing or devising (planning) the process.

The technical task of the invention is to provide a procedure which enables or improves multi-criteria optimization of a chemical or procedural process. Interaction between a user and an optimization tool is to be improved as well.

According to the independent claims, the problem is solved by a procedure, an interactive graphical user interface, a system or a computer program product. In a procedure for designing or devising a procedural or chemical process, interactive navigation can be performed on a display device.

At least a first and second configuration, preferably at least five configurations, can be available for selection. A first Pareto frontier is or can be formed for the first configuration. A second Pareto frontier is or can be formed for the second configuration.

A (corresponding) Pareto frontier is or can be formed for each of the configurations.

Based on the first Pareto frontier for the first configuration and the second Pareto frontier for the second configuration, a resulting Pareto frontier is or can be formed.

The resulting Pareto frontier can be shown or represented on a display device.

A first operating aid and a second operating aid can be shown on a display device, particularly in an operating area. The first operating aid can represent a first criterion and the second operating aid a second criterion. A setting or position of a selector of the first operating aid can represent a value of the first criterion. The setting or position of a selector of the second operating aid can represent a value of the second criterion. By moving or operating one of the selectors of the respective operating aid (e.g., first selector of the first operating aid or second selector of the second operating aid), the value of the associated criterion (e.g., first criterion associated with the first selector of the first operating aid or second criterion associated with the second selector of the second operating aid) can be changed.

The display device can be part of an input/output device.

Based on the position or setting of the selector (e.g., of one of the selectors or all selectors), a point or a section on the resulting Pareto frontier can be determined and/or shown.

In general, the point on the resulting Pareto frontier can be a value or a range of values that can yield the basis of the resulting Pareto frontier and a value of at least one criterion.

A selector can be a control element.

In a procedure for designing or devising a procedural or chemical process, a first Pareto frontier for a first configuration and a Pareto frontier for a second configuration is or can be provided. A resulting Pareto frontier can be determined or calculated on the basis of the first Pareto frontier and the second Pareto frontier.

The resulting Pareto frontier can be made available to a user; for example, it can be represented visually—or represented visually in such a way that the user can interact with the Pareto frontier or the representation of the Pareto frontier.

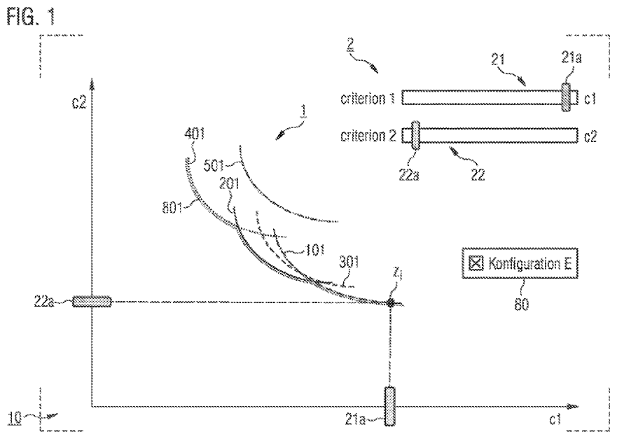

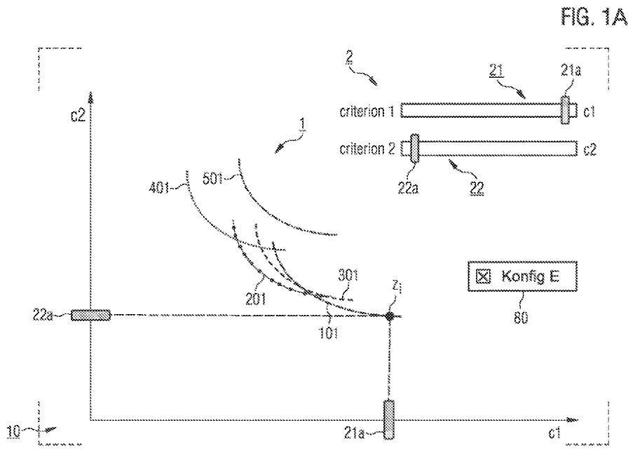

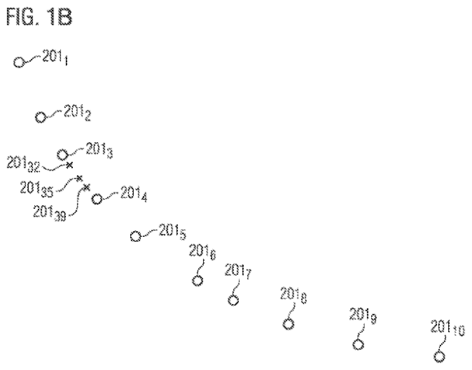

The following should be noted in order to understand the Pareto front(s) referred to above on several occasions. Each Pareto frontier is a function that is only functionally connected. Each of them has a large number of grid points. The frontier is therefore by no means to be understood as continuous but only as “functionally connected”, therefore a line is also shown in FIG. 1A for the grid points, which basically does not show a continuous Pareto frontier. This discrete function needs to be understood as present in FIG. 1. It can also be a projection from the n-dimensional space, in which space it exists, whereas it can only be shown graphically as a projection. The lines represent the functionally connected Pareto frontier. This is illustrated in enlarged form in FIG. 1B, with interpolation between the interpolation points.

The Figures serve as examples to assist with the understanding of the general statements.

Reachable points on the multidimensional Pareto fronts can be shown differently. Reachable points can be shown in light or contrasting colors. Points that cannot be reached, such as a projected surface, have a different contrast or color.

Color coding that is not shown can be used to represent a dimension that is not visible yet is still present. Uncertainties can be represented in five larger squares. A center of each of these would be the expected value, four arms in 90° orientations each indicate such areas of uncertainty as an interval.

A configuration can represent a procedural concept, such as a separation process or a mixing process.

A thermal separation process can be one configuration, a mechanical separation process another configuration and a chemical separation process yet another configuration. One of the processes can be selected for a separation task.

Different configurations can also be different thermal separation processes, such as rectification or drying.

A substance can also be a configuration, such as a reactant for a chemical reaction or a polymer component for a polyblend.

A Pareto frontier (also a Pareto set) is the set of all Pareto optima. Frequently, various objectives (criteria) can be defined for the optimization of a procedural or chemical process. For example, if a component is to be separated from a substance mixture, one criterion for the separation process can be the highest possible degree of purity of the separated component, and another criterion can be the lowest possible energy consumption of the process. These criteria are often contradictory, i.e., a high degree of purity of the separated component can only be achieved with a high energy input. The task of optimizing such a process involves multi-criteria optimization (also known as Pareto optimization).

For at least two configurations, a Pareto frontier can be created or provided for at least two criteria. A Pareto frontier can also be created or provided for at least three, or at least five, configurations for at least two criteria. The Pareto fronts can also be created or provided for more than two criteria (e.g., at least three criteria or at least five criteria).

In other words, there can be a first Pareto frontier for a first configuration and a second Pareto frontier for a second configuration. A resulting Pareto frontier can be formed from the Pareto fronts. The resulting Pareto frontier can be a non-dominated set of a union of Pareto fronts.

In other words, the resulting Pareto frontier can include or consist of Pareto optima of different Pareto fronts for configurations. For example, a first portion of the resulting Pareto frontier may correspond to a portion of a first Pareto frontier for a first configuration, and a second portion of the resulting Pareto frontier may correspond to a portion of a second Pareto frontier for a second configuration.

The resulting Pareto frontier can include or consist of different Pareto fronts for a particular configuration.

The resulting Pareto frontier can be shown as a curve in a two-dimensional diagram, with one axis for a first criterion and the other axis for a second criterion. The resulting Pareto frontier can also be a (curved) surface in a three-dimensional diagram (shown in two dimensions), with three criteria for the axes. A star diagram can also be used to represent the resulting Pareto frontier. A three-dimensional representation of the resulting Pareto frontier is possible as well.

A number of operating aids can be shown in the operating area of the display device, each of them representing one criterion. The value of the criterion is changed when a selector of a respective operating aid is moved or operated. At least some of the operating aids can be shown as sliders. In most cases, a movement to the left results in an improvement. Changing the value of a criterion (by the selector of the operating aid) changes the values of other criteria (represented by a change in the setting or position of the selector of the other operating aid or operating aids). The values of other criteria can change on the basis of the resulting Pareto frontier.

Based on the values of the criteria (settings or positions of the operating aid selectors), a point is determined on the resulting Pareto frontier and preferably shown on the display device.

A line or surface can also be defined, with the point lying on the line or surface.

A criterion can represent a physical or chemical quantity.

The selectors form (and enable) interactive communication with a user. A solution selected in the current state is shown as a point (zᵢ) on the resulting Pareto frontier. The point and the operating area are functionally linked or coupled with each other. What is selected in the operating area is also displayed on the resulting Pareto frontier. If the selected solution in the operating area is changed when the planner changes the position of a respective selector of a respective operating aid, the point (zᵢ) moves along the resulting Pareto frontier.

The position of the selector of an operating aid can be changed linearly (as a slider) or as a rotational position (rotational specification). The operating aids can be displayed on the display device and operated using a mouse pointer or directly with the finger of one hand on a touch-enabled screen (touchscreen).

With the slider, the operating aid can be linear. For an operating aid with a variable rotary position, the operating aid can have a curved adjustment range. As a rule, the operating aids are depicted in two dimensions, but this does not limit their understanding.

The value of the respective criterion can be increased by moving or operating one of the selectors in one direction. The value of the criterion can be reduced by moving or operating one of the selectors in the opposite direction.

By moving or operating one of the selectors, the position or setting of the other selector can be changed. This specifically occurs without any input on the part of the user. Preferably, the position or setting of the other selector is changed on the basis of or along the resulting Pareto frontier.

In the procedure, it can be determined whether a deviation or difference between the value for the second criterion of the first Pareto frontier for the first configuration and the value for the second criterion of the second Pareto frontier for the second configuration exceeds a threshold value. The determination is preferably done with an equal value of the first criterion. This makes it possible to check whether two configurations can be considered equivalent.

Specifically, it can be determined whether a deviation or difference between the value for the second criterion of the resulting Pareto frontier and the value for the second criterion of the first and/or second Pareto frontier for the first and/or second configuration exceeds a threshold value. The determination is preferably done with an equal value of the first criterion. This makes it possible to check whether two configurations can be considered equivalent.

The resulting Pareto frontier represents the set of Pareto optima based on the selection of values for the criteria, which in turn is based on the Pareto fronts of the configurations. If there is only a small or justifiable difference (deviation below the threshold value) between two configurations, they can be regarded as equivalent or comparable solutions for multi-criteria optimization, with this signaled to the user.

The threshold value can be definable or defined by a user. Preferably, a threshold value selection can be shown on the display device with a selector. The threshold value can be definable or defined by the selector of the threshold value selection.

Specifically, the threshold value is a maximum relative deviation, such as in percent. The threshold value can also be an absolute value.

If the threshold value is exceeded, at least two selectors can be shown on the second operating aid. The position or setting of one of the selectors can represent or show the value of the second criterion of the first Pareto frontier for the first configuration. The position or setting of the other of the selectors can represent or show the value of the second criterion of the second Pareto frontier for the second configuration.

The number of selectors shown on an operating aid can correspond to the number of configurations that were determined as equivalent solutions for multi-criteria optimization.

If the threshold value is not exceeded, no more than one selector can be shown on the second operating aid, i.e., exactly one selector. The position or setting of the selector can represent or show the value of the second criterion of the resulting Pareto frontier.

An interval is or can be defined for the value of the first and/or second criterion. The interval can be a blur interval.

The interval can be defined by manipulating a selector. For example, the selector can be selected and the interval defined by operating a scroll wheel of a mouse or by a finger input of the user (e.g., pulling two fingers apart on a touchscreen).

One or more configurations can be determined in the process. The configurations determined can be those whose Pareto frontier for the respective configuration includes a point that lies within the interval for the first criterion and relates to a value of the second criterion that is derived from the value of the first criterion and the resulting Pareto frontier.

The one or more configurations determined can be output, shown or displayed.

The interval can be a first interval for the value of the first criterion, and a second interval is or can be defined for the value of the second criterion.

In particular, the interval is a blur interval.

One or more configurations may be determined, whereby the Pareto frontier for the respective configuration includes a point that lies within the first interval for the first criterion and within the second interval for the second criterion.

One or more configurations can be output, shown or displayed.

The first interval and/or second interval can be or can be defined by a user.

A score (e.g., a number of points) can be determined for the specific configurations, whereby the score represents the suitability of the associated configuration.

The score can be output, shown or displayed.

The suitability of the configuration can result from the deviation of the Pareto frontier of the configuration from the resulting Pareto frontier, in particular within the interval or intervals. The score can be higher if the deviation is lower.

The score of the respective configuration can refer to a selected configuration. To do this, the user can select a configuration to which the score is to relate, in particular on the display device.

When determining the score of the respective configuration, one of the following characteristics (influencing variables) can be taken into account, and several of the following characteristics can also be taken into account
A difference or deviation of points on different Pareto fronts of the configurations relative to one another, in particular points with reference to a mean value of the first and/or second interval
A curve gradient of the Pareto frontier within the first and/or second interval
A certainty in the determination of the Pareto frontier, in particular a certainty in the determination of the values of the Pareto frontier
A number of setting options of the configuration One or more configurations can be (temporarily) excluded or excludable. Specifically, one or more configurations can be (temporarily) excluded or excludable by a user.

The excluded configuration(s) cannot be included in the determination or calculation of the resulting Pareto frontier.

It can be determined whether the exclusion of one or more configurations changes the value range of the resulting Pareto frontier. Specifically, it can be determined whether the exclusion of one or more configurations reduces the value range of the resulting Pareto frontier.

A change in the value range of the resulting Pareto frontier can be signaled to the user. Specifically, this is or can be shown on the display device. This is or can preferably be shown in the operating area of the display device.

At least three, preferably at least four, more preferably at least five, even more preferably at least six, most preferably at least eight, Pareto fronts can be formed for the same number of configurations. The resulting Pareto frontier can be formed on the basis of all the Pareto fronts formed for the configurations.

The resulting Pareto frontier can be a non-dominated set of a union of the Pareto fronts, in particular all Pareto fronts.

Different sections of the resulting Pareto frontier can correspond to different Pareto fronts of configurations. Specifically, different proportions of the resulting Pareto frontier can be different proportions of Pareto fronts for configurations.

An interactive graphical user interface may be provided for designing or devising a procedural or chemical process.

A resulting Pareto frontier can be formed at least on the basis of a first Pareto frontier for a first configuration and a second Pareto frontier for a second configuration.

At least a first interactive operating aid and a second interactive operating aid can be shown on a display device in an operating area. The first operating aid can represent a first criterion and the second operating aid a second criterion. A position or setting of a selector of the first operating aid can represent a value of the first criterion, and a position or setting of a selector of the second operating aid can represent a value of the second criterion.

The value of the respective criterion can be changed by a user by moving or operating one of the selectors of the respective operating aid.

A point on the resulting Pareto frontier can be determined and/or shown based on the position or setting of the selector defined by the user.

A resulting Pareto frontier can be shown on the display device.

The display device can show which of the Pareto fronts for the respective configuration determines the section or part of the resulting Pareto frontier on which the point lies or is shown.

An interactive threshold value selection can be shown on the display device with a selector. A value for a threshold can be changed by moving the selector. If a deviation or difference between a value for the second criterion of the first Pareto frontier for the first configuration and the value for the second criterion of the second Pareto frontier for the second configuration exceeds the threshold value, in particular if the value of the first criterion is the same, at least two selectors can be shown on the second operating aid. The position or setting of one of the selectors may represent or show the value of the second criterion of the first Pareto frontier for the first configuration, and the position or setting of the other of the selectors may represent or show the value of the second criterion of the second Pareto frontier for the second configuration.

If the threshold value is not exceeded, no more than one selector (e.g., exactly one selector) can be shown on the second operating aid. Specifically, the position or setting of the selector can represent or show the value of the second criterion of the resulting Pareto frontier.

An interval for the value of the first and/or second criterion is definable on the interactive graphical user interface.

The interval can be a blur interval.

The interval can be shown or represented on the display device in the area of the resulting Pareto frontier and/or in the area of the first Pareto frontier for the first configuration and the second Pareto frontier.

The interval can be shown or represented on the display device in the area of the first and/or second operating aid.

A selection of one or more configurations can be shown on the display device. A user can exclude or include one or more of the configurations. The resulting Pareto frontier can be based on Pareto fronts for configurations that are included. Specifically, the resulting Pareto frontier cannot be based on Pareto fronts for configurations that are excluded. Included Pareto fronts can be ones that are selected. Excluded Pareto fronts can be ones that are not selected or have been deselected.

A change in the value range or a remaining value range of the resulting Pareto frontier can be shown on the display device. This is especially the case if the exclusion of one or more configurations changes the value range of the resulting Pareto frontier.

A system may include a processor unit (processor). The processor unit may be configured in such a way to perform a procedure disclosed there. The processor unit may be configured to generate an interactive graphical user interface disclosed there.

A computer program product can include commands. When the computer program product is executed by a processor or computer, the instructions may cause the processor or computer to perform a procedure disclosed there. Moreover, when the computer program product is executed by a processor or computer, the instructions may cause the processor or computer to generate an interactive graphical user interface disclosed there.

In one procedure, an interactive graphical user interface can be created, which in particular can be shown on a display device.

The interactive graphical user interface may be an interactive graphical user interface disclosed there.

A processor can be designed to create a resulting Pareto frontier at least on the basis of a first Pareto frontier for a first configuration and a second Pareto frontier for a second configuration.

The processor can create an operating area for the interactive graphical user interface with at least a first interactive operating aid and a second interactive operating aid, whereby the first operating aid represents a first criterion, and the second operating aid represents a second criterion. A position or setting of a selector of the first operating aid can represent a value of the first criterion, and a position or setting of a selector of the second operating aid can represent a value of the second criterion.

Based on a user's input, such as at an input device or an input/output device, the processor can determine a point on the resulting Pareto frontier.

The input can be a movement or operation of one of the selectors of the respective operating aid, thereby changing the value of the respective criterion.

The point on the resulting Pareto frontier can be determined (by the processor) on the basis of the user-defined position or setting of the selector.

The processor can determine which of the Pareto fronts for the respective configuration determines the section of the resulting Pareto frontier on which the point lies.

Based on an input of a threshold value by the user, the processor can determine whether a deviation or difference between a value for the second criterion of the first Pareto frontier for the first configuration and the value for the second criterion of the second Pareto frontier for the second configuration exceeds the threshold value, especially if the value of the first criterion is the same.

If the deviation or difference exceeds the threshold value, the processor can determine at least two selectors for the second operating aid, whereby the position or setting of one of the selectors represents the value of the second criterion of the first Pareto frontier for the first configuration, and the position or setting of the other of the selectors represents the value of the second criterion of the second Pareto frontier for the second configuration.

If the deviation or difference does not exceed the threshold value, the processor cannot determine more than one (exactly one) selector for the second operating aid. The position of the selector can represent the value of the second criterion of the resulting Pareto frontier.

Based on an input from the user of a first interval (specifically a fuzziness interval) for the first criterion and/or a second interval (specifically a fuzziness interval) for the second criterion, the processor can determine whether one or more configurations includes a Pareto frontier that includes a point within the first interval for the first criterion and/or within the second interval for the second criterion (c2)

Based on a selection by the user, the processor can use configurations selected by the user to determine the resulting Pareto frontier.

Based on a selection by the user, the processor can exclude configurations deselected by the user from the determination of the resulting Pareto frontier.

Based on the user's selection, the processor can determine whether a change in the value range of the resulting Pareto frontier exists or is determined.

Embodiment examples of the invention are explained in more detail with the aid of the Figures. All explanations are equally applicable to the disclosure, but they are not to be interpreted in such a way that they must be included as necessary elements of the claims. All of the following examples remain examples even if they are not explicitly preceded by “for example”.

FIG. 1 is a schematic view of a sample sequence of a process of one or more embodiments of the invention. Here are configurations on a display device with display 10.

FIG. 1A helps with understanding the Pareto frontier—in the example of Pareto frontier 201. A large number of grid points can be seen. The frontier is therefore by no means continuous, as FIG. 1 suggests. It is “only” functionally connected, which is why it is also shown as a (thin) line in the following FIG. 1B, which is not really a line. It basically consists of discrete grid points. The Pareto frontier can also be thought of as a projection on the n-dimensional space in which it exists, whereas it can only be visualized graphically as a projection.

FIG. 1B also helps to understand the Pareto frontier, in the example of Pareto frontier 201. A large number of grid points from 201₁ to 201₁₀ can be seen. Three support points 32, 35 and 39 are planned between 201₃ and 201₄. The same can be stipulated between other (neighboring) grid points.

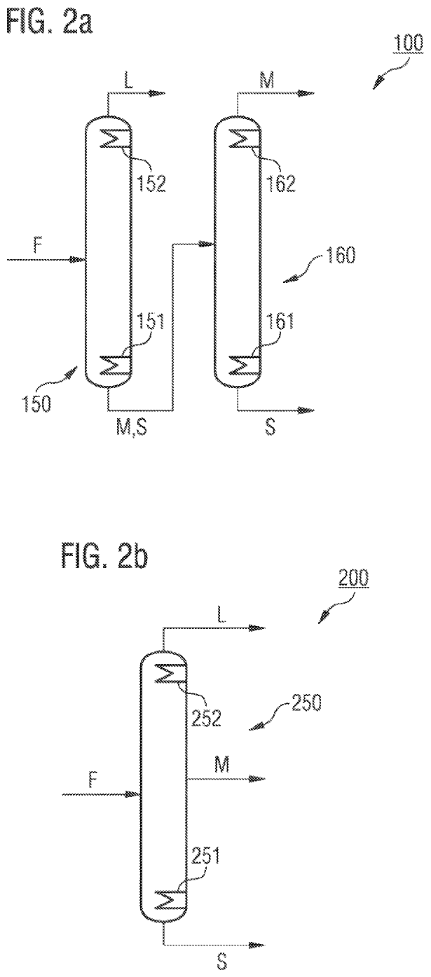

FIGS. 2a

and 2b show two configurations 100 and 200.

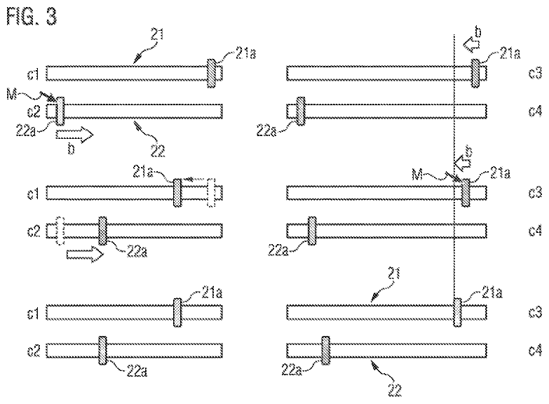

FIG. 3 illustrates a diagram of the movement of selectors 21a and 22a of operating aids 21 and 22 in several image sections.

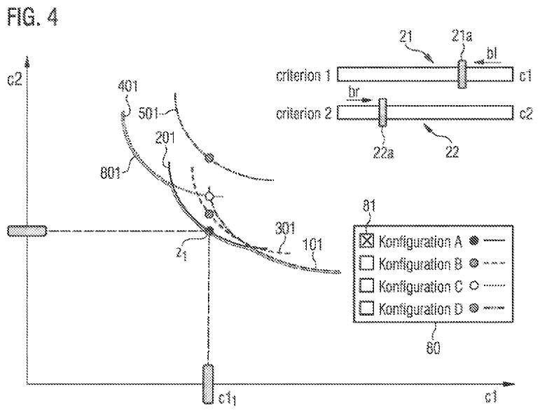

FIG. 4 illustrates an advanced system state on display device 10 with a display, based on the system state in FIG. 1.

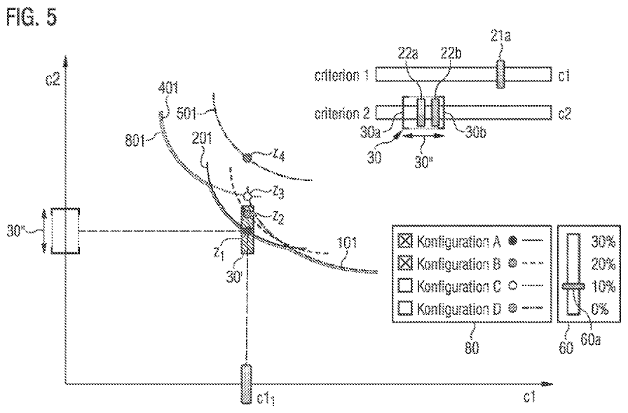

FIG. 5 illustrates another system state in which a similarity area 30′ has been opened at value c1₁. The similarity area is circumscribed here by a (vertical) strip 30′, and its length 30″ corresponds on the second axis as second criterion c2 to the section between ends 30a and 30b. A threshold selection 60 is also shown.

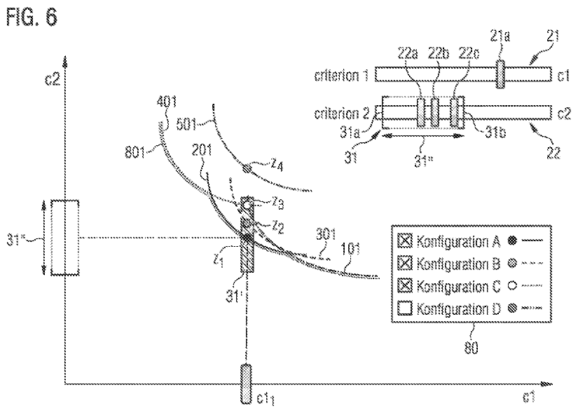

FIG. 6 is yet another system state in which the similarity area has been extended to a longer field 31′, corresponding to the increased length 31″.

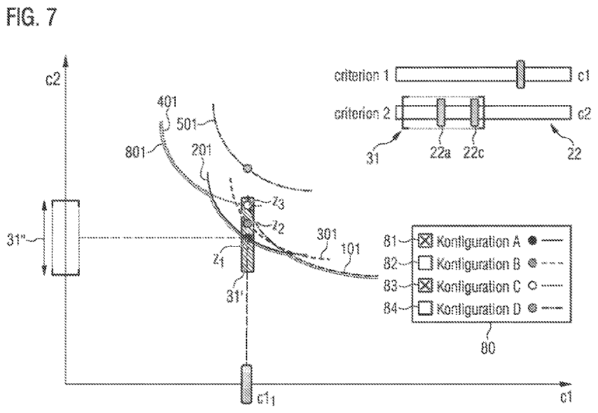

FIG. 7 illustrates the system state of FIG. 6, whereby one of the configurations has been deselected via a visual, interactive field 80 as a function field, so that this configuration no longer appears or is no longer displayed in operating area 2.

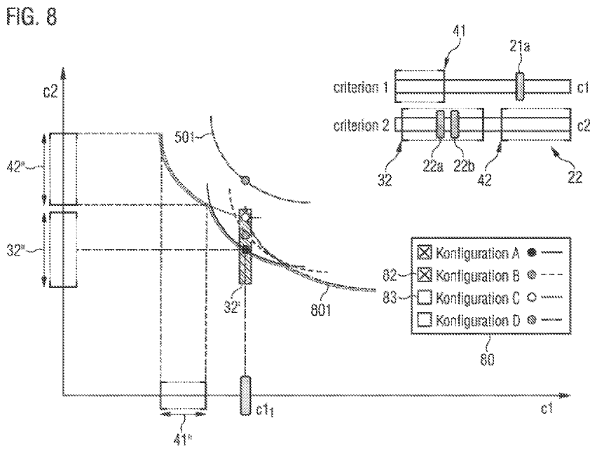

FIG. 8 illustrates the system state of FIGS. 6 and 7, with the exception of configuration C, which leads to the restriction of selection options labelled with the areas 42″ and 41″.

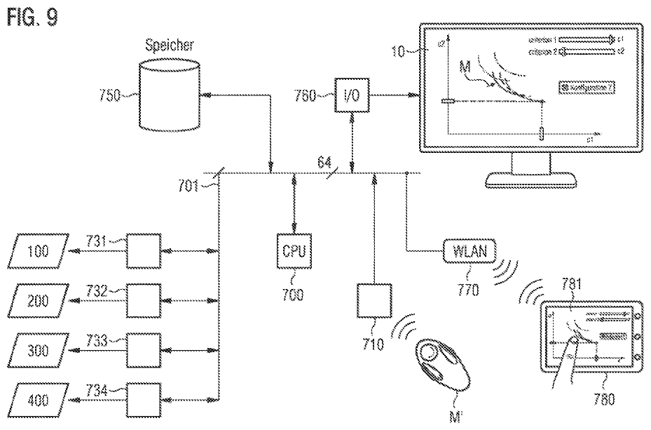

FIG. 9 illustrates a technical-functional data system.

FIG. 1 illustrates an input/output device with a display device 10 (e.g. display). This display device 10 is symbolized by the four corners indicated. It also results from the system structure of FIG. 9 with which the functions described below are achieved (or are achievable).

FIG. 1, enlarged, shows an operating area 2 at the top right and a configuration area 1 on the left, which takes up more space than operating area 2. Operating area 2 is outside configuration area 1, which means that the two do not overlap functionally. They appear visibly separated from each other for the planner, whereby their position is only selected in the example in FIG. 1 in such a way that the configuration area is arranged at the bottom left and the operating area at the top right (on the visible surface of display device 10).

In general, operating area 2 is or can be shown on display device 10. In particular, operating area 2 is or can be shown on display device 10 without configuration area 1.

Configuration area 1 with its associated elements cannot be shown on display device 10.

Operating area 2 and configuration area 1 do not need to be shown together. Operating area 2 and configuration area 1 can be functionally uncoupled or functionally decoupled.

There is a visible and visually delimited field 80, which identifies configuration E as a functional field. This field, which in general is also called function field 80, is explained and expanded upon later.

Operating area 2 and configuration area 1 can be functionally linked to one another. This functional coupling bears explaining. Several Pareto fronts for different configurations are shown in configuration area 1; in the example, these are Pareto fronts 101, 201, . . . to 501. A resulting Pareto frontier 801 is also shown.

Resulting Pareto frontier 801 is based on sections of different Pareto fronts for configurations. In the example of FIG. 1, resulting Pareto frontier 801 contains sections of Pareto fronts 401, 201 and 101. Resulting Pareto frontier 801 is the set of Pareto optima (Pareto set) of the configurations. Resulting Pareto frontier 801 therefore represents the Pareto optima of the considered Pareto optima of the configurations.

In general, resulting Pareto frontier 801 in the Figures is slightly offset graphically from the Pareto fronts of the configurations for better clarity. This can also be shown on a display device 10.

Also shown in configuration area 1 are two axes c1 and c2, which are perpendicular to each other and represent two criteria c1 and c2. Criterion 1 (also c1) and criterion 2 (also c2) are shown, which are visible and navigable in operating area 2.

In another representation, more than two axes can be shown for criteria, in particular three axes or more than three axes (e.g., in a network diagram or star diagram). The representation can also be based on a projection or projections if, for example, several criteria are to be shown.

An intersection can be found at the currently selected point zᵢ. This selected point results from the settings of both operating aids 21 and 22, which can be seen in operating area 2. Criterion 1 is also called c1 and is plotted horizontally in configuration area 1. Criterion 2, also known as c2, is plotted vertically in configuration area 1.

In operating area 2, two settings are assumed for the two criteria c1 and c2 and with the two operating aids 21 and 22; these settings can be selected using selectors 21a and 22a and are also shown selected here. This setting results in the selected point zᵢ of resulting Pareto frontier 801 and Pareto frontier 101 of configuration E when operating area 2 is functionally coupled with configuration area 1, since the section of resulting Pareto frontier 801 on which point zᵢ is located is formed by Pareto frontier 101 of configuration E.

The concept of the Pareto frontier also bears explaining. The Pareto frontier is a representation of all technical variables that represent a configuration and, by definition, cannot be improved by multiple criteria. Such a multi-criteria optimum is characterized by the fact that there are no better solutions than those that lie on the Pareto frontier. No value (criterion) can be improved without worsening another criterion.

It can be seen that each additional point on Pareto fronts 101, . . . , 501, 801 also represents a solution, so that the concatenation of all solutions on a Pareto frontier defines the curve of this Pareto frontier. If the user (as the user of the tool for designing and configuring a procedural or chemical process according to FIG. 1) changes the configuration, another procedural concept, another (chemical) raw material or another (similar) reactant is or can be selected.

For example, a first procedural concept is a first configuration and a second procedural concept is a second configuration, or a first (chemical) raw material is a first configuration and a second (chemical) raw material is a second configuration, or a first (similar) reactant is a first configuration and a second (similar) reactant is a second configuration. The user can change the “optimum” configuration by changing the values of the criteria.

Setting the two selectors 21a and 22a defines point zᵢ in configuration area 1. The settings of selectors 21a and 22a are still represented on the two criterion axes c1 and c2, the selected x-y representation clearly indicates point zᵢ on resulting Pareto frontier 801, whereby this section of resulting Pareto frontier 801 corresponds to a section of Pareto frontier 101 of configuration E. This shows that configuration E is the best solution for the selected values of criteria c1, c2.

Configurations symbolized by A, B, . . . E can be shown.

According to the previous description, operating area 2 includes two interactive operating aids 21, 22, whereby each operating aid is (currently) assigned a selector 21a or 22a. Examples of operating aids are sliders; examples of selectors are digitally displayed control buttons.

This selector can be used to improve a respective criterion in operating area 2 or to worsen it in the corresponding opposite direction. According to existing conventions, a movement to the left is an improvement, so that criterion 2 on c2 is already fully optimized (as it is on the left edge of operating aid 22). For the example on the next page, the “selector of the slider is close to the left stop”.

In FIG. 1, the corresponding illustration is shown in configuration area 1, where selector 22a has the lowest value. Increasing the value of criterion c2 in the design or devising (planning) leads to a deterioration of this criterion, but since the criterion c1 must also be observed and can still be improved on the basis of the position of selector 21a, a movement is to be expected, which is explained with the aid of the following Figures.

In an overview, selector 21a moves to the left (also to the left on axis c1). Selector 22a moves to the right (upwards on axis c2). The orientation of operating aids 21 and 22 in operating area 2 can also be swiveled by 90° so that left/right becomes down/up. This can also apply to each of the selectors, which are aligned in such a way that they appear like a Cartesian coordinate system.

This movement is explained in individual steps in FIG. 3 in six image sections; more on this later.

FIGS. 2a and 2b show two procedural concepts as examples to illustrate this. Each of the concepts is a configuration.

Substance mixture F is present, which (simplified) consists of a low boiler L (e.g., hexane), a medium boiler M (e.g., heptane) and a high boiler H (e.g., octane) as components. Light boiler L, medium boiler M and high boiler H are present equimolar in substance mixture F of the example.

Substance mixture F is to be separated into its components. A first criterion is the degree of purity of medium boiler M (heptane), which needs to be as high as possible. A second criterion is the cost of separation, whereby the cost takes into account the equipment/engineering cost and the energy cost. The second criterion is that the cost is to be as low as possible.

In general, the task may be subject to restrictions or constraints. In the example given, it may be the case that a minimum degree of purity of 99 mol % is specified for both low boiler L and high boiler H.

The first configuration 100 includes a first rectification column 150 and a second rectification column 160. The first rectification column 150 includes a first heat exchanger 151 (evaporator) at the bottom and a second heat exchanger 152 (condenser) at the top. The second rectification column 160 in turn includes a first heat exchanger 161 (evaporator) at the bottom and a second heat exchanger 162 (condenser) at the top.

In the first rectification column 150, the main top product is light boiler L, and the main bottom product is a mixture of medium boiler M and high boiler H.

The bottom product of the first rectification column 150 is fed to the second rectification column 160. The main top product of the second rectification column 160 is medium boiler M, and the main bottom product is high boiler H.

The second configuration 200 includes a rectification column 250 with a first heat exchanger 251 (evaporator) at the bottom and a second heat exchanger 252 (condenser) at the top. The main bottom product of this configuration 200 is high boiler H and the main top product is light boiler L. As the main product, medium boiler M is removed via a side discharge.

In general, the two-column concept (configuration 100) can achieve a higher degree of purity of medium boiler M than the column concept with a side discharge (configuration 200), but the expense, in particular the engineering/equipment expense, of the two-column concept is usually higher than that of the column concept with a side discharge.

A Pareto frontier can be formed for each of configurations 100 and 200. A resulting Pareto frontier can be formed from these Pareto fronts. A user can now use the resulting Pareto frontier to check which configuration is the most suitable for the selected compromise of criteria (purity, mean value M and expense).

If the user wants a very high degree of purity of medium boiler M, the user can set the value for this criterion accordingly, such as at a selector 21a, as shown above. The second selector 22a moves to an unfavorable value for the second criterion (expense) according to resulting Pareto frontier 801. The user recognizes that a high degree of purity of medium boiler M is only possible at great expense. The user can also recognize, if applicable, that the desired degree of purity of medium boiler M is only possible with one of the configurations, such as configuration 100 in this example.

Similarly, a very low cost is only achievable if the degree of purity of the medium boiler M is relatively low. As a rule, the expense required for single-column concepts (here with a side discharge) is lower.

In a range in which the desired degree of purity of medium boiler M can be achieved by both configurations 100 and 200, selected point zᵢ on the resulting Pareto frontier lies on a section of the same ones, determined by the Pareto frontier of the configuration that has the lower expense (value of the second criterion).

The user can therefore have an overview of the entire spectrum of possible values for the criteria, while at the same time the user can be quickly and intuitively shown which of the configurations is superior to the other configuration or configurations for a specified criterion (Pareto-optimal).

FIG. 3 illustrates the change in selectors 21a and 22a of the two criteria c1 and c2 in slow motion or in small steps. Three image sections in the left half of FIG. 3 are representative of this. A function pointer M is assumed, which is explained in greater detail later.

Starting from a setting of selectors 21a and 22a on operating aids 21 and 22, the initial state depicted in FIG. 1 is shown. Criterion c2 is set to the best possible value, as it is furthest to the left. In the example, worsening of this value is to be brought about by movement b, whereby control element 22a is to be moved to the right by a mouse pointer M (the arrow element) or, in the case of a touchscreen 780 according to FIG. 9, by activating and swiping with a finger. It moves from its original location in the upper half of the center display to a location further to the right. This can be shown using an optical fading technique; the previous location fades and the new location appears.

Movement b is completed or finished when selector 22a has reached its new position, as shown in the third image section on the left. The original position, which was still visible in the center image section, can no longer be seen. In one movement from the first to the third image section, there are many intermediate states that are not shown here.

At the same time as selector 22a is moving in one direction, selector 21a of the other criterion c1 can be moved in the other direction, preferably supported by the procedure, determined by a processor for example. In particular, the movement of the other criterion c1 is orientated towards the resulting Pareto frontier. Criteria c1 and c2 (selectors 21a and 22a) can be functionally linked.

The actual functional connection takes place through specification of the predefined solutions on the Pareto frontier (resulting Pareto frontier); if the user changes a solution in one criterion, a second, third and/or fourth criterion also changes, and thus also the second (or other) selector or selectors in precisely this second criterion or for precisely this criterion c1.

FIG. 3 shows two other criteria c3 and c4 in the right half. Here you can see a movement of criterion c3 (selector 21a) from right to left, i.e., towards a better value. Again, selector 22a moves according to the change or movement of selector 21a, specifically based on resulting Pareto frontier 801.

Movement b is shown running slowly and gradually up to the marked vertical line and the new position of selector 21a on operating aid 21. Function pointer M (the arrow element) takes selector 21a of operating aid 21—under the user's control—and sets it to the position of the third image section.

Taking into account the options that several criteria can be changed via operating aids, although only two criteria c1 and c2 are explained in detail in the examples selected here, it can also be understood with the aid of FIG. 3 that two further criteria c3 and c4 can be changed—or that criteria c1 and c4 are shown in operating area 2, functionally coupled with the representations of Pareto fronts 101 to 501 and resulting Pareto frontier 801 (in axes c1 and c4) that are then fitting in configuration area 1. Typically, five to six criteria are compared. The Pareto calculations for specifying the predefined solutions use between ten and fifteen criteria. There are three to five criteria in mutual competition, between which a compromise must be found in order to find and define a solution with which the user is sufficiently satisfied.

In FIG. 4, the user has set an improved value for criterion c1 by moving selector 21a to the left. Due to the functional coupling and the curve of the Pareto fronts, there is a shift to the right for criterion c2, whereby selector 22a is automatically shifted to the right via the functional coupling, while selector 21a is shifted to the left by the user, such as by clicking with the mouse or by touching and swiping/sliding.

The two arrows br and bl show this movement. The movement was illustrated in FIG. 3, where the functional coupling was eliminated and only one selector moved at one criterion.

FIG. 4 shows that, by moving selector 21a of criterion c1 to an improved value, point z₁ controlled in configuration area 1 is a different one. By moving control element 21a to the left, point z₁ on resulting Pareto frontier 801 (a section of Pareto frontier 201) moves from further down on the right to further up on the left. In the example, this is represented at the point where the two selectors 21a and 22a come to rest on criteria c1 and c2 in operating area 2.

What can be better understood with the aid of FIG. 1 in FIG. 4 is the movement of the generally positioned point zᵢ towards the point zᵢ shown here. The points lie on sections of the resulting Pareto frontier 801 that are caused by different Pareto fronts for configurations, namely the section on resulting Pareto frontier 801 on which point zᵢ lies by Pareto frontier 101 and the section on resulting Pareto frontier 801 on which point z₁ lies by Pareto frontier 201.

FIG. 4 then symbolizes as well that, at point c11, which is held by selector 21a in operating area 2, there are several points that lie on other Pareto fronts, such as the points on Pareto fronts 301, 101 (or 401) and 501. However, since the associated configurations are not selected, configuration A, which is assigned to Pareto frontier 201, is retained. What this conveys is that the section on resulting Pareto frontier 801, on which the point zᵢ is located, is determined by Pareto frontier 201 of configuration A.

This defines function field 80, which represents a number of configurations that can be selected and deselected by the user. This selection is symbolized by an x in checkbox 81. The assignment of the points is also symbolized by the appropriate coloring and the associated Pareto frontier on which they can move.

The configurations to be displayed can be specified in function field 80 as a visual field.

However, the selection of further configurations does not mean that multiple configurations can be selected in FIG. 4 or that multiple configurations are therefore available for a setting or can be selected for navigation. To do this, an extension can be effected from a coordinate to a single target variable (a criterion) to an interval on at least one of the axes.

This setting is shown in FIG. 5. The setting shows the same state as that of FIG. 4 with the three possible target points zᵢ, z₂, z₃ or z₄. Two of these four possible configurations are selected in function field 80, so that two configurations lie in the similarity area of a blur interval 30, which extends the single coordinate of criterion c2 to a coordinate area. The extended range of length 30″ is the interval or blur interval (also blur range) with a lower border 30a and an upper border 30b. Since the convention requires an improvement to the left, the terms upper and lower range are misleading, so they should be called left end 30a and right end 30b instead. The blur range 30 extends between these two with a length of 30″.

The blur interval on criterion c2 is also shown graphically in configuration area 1, on criterion axis c2.

The length 30″ corresponds to the interval between left end 30a and right end 30b in operating area 2. This area is also shown via the vertically extending field 30′, which in the example shown covers two configurations: configuration A and configuration B.

Selecting these two configurations in function field 80 gives the user an additional option. Instead of just configuration A along Pareto frontier 201, configuration B, which runs along Pareto frontier 301, can also be selected, as it is very similar to configuration A and Pareto frontier 201.

The interval can be defined by a threshold value. For example, if a value is defined for the second criterion, such as by the position of selector 22a, a relative deviation can be determined, e.g., 10%. The value and the threshold value yield an interval. The threshold value can be regarded as a deviation by the value of the criterion. As a specific example, a value of 50 can be set for the second criterion, with 10% set as a threshold value. The interval can then be 50±10% (i.e., 50±5).

The threshold value can also be specified as an actual interval, such as a value of 45 to 55 for a criterion.

It is also possible to specify an absolute value of the deviation around a value of a criterion. For example, an absolute deviation of 5 (five) can be specified as the threshold value, resulting in one of 50 for the criterion 50±5.

The interval can be specified asymmetrically around a value, such as around a specified value of the criterion. For example, a threshold value can be defined for a criterion for a value of 50, resulting in an interval of 48 to 58.

The threshold value can be specified by a user, such as via a threshold value selection 60 as shown in FIG. 5. Threshold selection 60 may include a threshold selector 60a. Threshold value selector 60a can be moved, with the threshold value being increased by movement in one direction and reduced by movement in the opposite direction.

Alternatively, it can be defined by clicking the mouse or by using the scroll function of a mouse or by double-tapping and swiping on a tablet, i.e., with a touch-sensitive output/input device 780 as the input/output device of display device 10.

Threshold values or intervals can be specified for at least two criteria. In particular, threshold values for the intervals is or can be specified for all criteria.

In particular, threshold values or intervals is or can be specified differently for different criteria.

The term “function pointer” is used across all devices, combining the pointer and function option. With an icon, the mouse indicates that holding down a click on the display allows a “Drag” function, while a double-click allows a selection. Similarly, the single tap or double tap of a finger works as a locator or activator on a screen-enabled or touch-enabled device (e.g., a tablet). The same applies to the laser pointer as a pointing device, which is linked to a function by on/off or the sign of an icon at the pointing location, e.g., as a small circle with a 360° movement in a clockwise or anti-clockwise direction.

Also shown in FIG. 5 is the appearance of the second Pareto frontier 301 and the further point z₂ covered by the blur by showing a second selector 22b. Both selectors 22a and 22b lie within the two borders 30a, 30b of the extended identity or similarity 30″.

A deviation between points zᵢ and z₂ is therefore below the threshold value, or points z₁ and z₂ lie within the interval (blur interval).

It has already been mentioned that field 30′ extended to length 30″ as a blur interval 30 in configuration area 1 leads to narrow field 30′, shown with hatch marks here, which covers at least two Pareto fronts. Since the user also controls the range or the size of the blur, the user can achieve 30 by narrowing and expanding this blur interval and can also see interactively whether there is any leeway in the designing or devising process. If the blurring is too low (low threshold value or small interval), no further selector appears in its control panel. If the blur is larger (larger threshold value or larger interval), as in the example shown with the two ends 30a and 30b, at least one further configuration is available for selection, as can also be seen in function field 80.

The configuration corresponding to Pareto frontier 401 or Pareto frontier 101 or Pareto frontier 501 cannot be achieved, as associated target points z₃ und z₄ lie outside blur interval 30′. They are therefore above the threshold value or outside the interval.

After specifying the criteria and defining a list of possible configurations, the Pareto fronts shown in the Figures can be calculated automatically.

The user is offered all configurations (Pareto fronts) for simultaneous navigation and, by means of interactive operability, has the option of leaving the identity area in favor of a similarity area, called blurring, in order to have multiple configurations to choose from at the same time. To this end, it is also evident that the user can switch from one configuration to the next, i.e., from one Pareto frontier to another Pareto frontier, and is therefore not restricted to one configuration and one Pareto frontier. This makes it possible to have a non-dominant set of the union of all Pareto fronts of the configurations available for navigation (resulting Pareto frontier).

In a practical sense, FIG. 5 would mean for a user that a procedural or chemical process can only be performed with the configuration corresponding to Pareto frontier 201 (point z₁)—but also with configuration B of Pareto frontier 301 in blur interval 30.

This is particularly relevant for the user if further criteria are available for selecting a configuration. For example, the user or the user's company may have more experience with one procedural concept as a configuration than with another procedural concept as a configuration. If both configurations have been assessed as similarly usable, for example, the configuration with which the user or the user's company has more experience can be selected.

Function field 80 can also have the function of a preview, as shown in FIG. 5, in which four configurations A to D are offered in function field 80.

These multiple, here four, configurations are the ones that are possible via value c1₁ because they are all reached by this value, i.e., target points z₁ to z₄. All these configurations appear automatically controlled by the associated software in extended function field 80, compared to configuration E from FIG. 1. Point zᵢ there and the position of selector 21a do not permit any additional intersection points, so that no additional Pareto fronts can be selected, unlike in FIG. 5, in which the position of selector 21a offers four possibilities, albeit with a different blur interval 30.

FIG. 6 illustrates the system state of FIG. 5, and an extended field 31′ is specified, which is created by moving two borders 31a and 31b in operating aid 22. The span of this extended similarity area 31″, as also shown on axis c2 of the second criterion, is larger than the span 30″ in FIG. 5.

More Pareto fronts are achieved, and there are more intersections z₁, z₂ and z₃. These points, target points or intersections are also displayed interactively and visually on the operating aid or in the operating aid for criterion c2. Two additional selectors 22b and 22c are created there with existing selector 22a, which represents intersection point zᵢ.

The extended similarity area extends the first similarity area of FIG. 5. FIG. 5 itself was already a first area of similarity that went further than the identity of the figure in FIG. 4 in order to cover multiple configurations. All these extensions, which go beyond a single coordinate, are conceptualized by the blur as an interval (threshold value), which continues to be controllable in itself, as described below.

Blur interval 31 with extension 31″ due to vertical field 31′ at target value c11 of criterion axis c1 can also be changed, as already explained above; it can be made larger or smaller, which is something the user can do. This change can be made by clicking and dragging borders 31a or 31b, individually or together. It can be done on an input/output device (e.g., touchscreen) 780 by activating a border with an initial touch (tap) and then moving it with a swipe.

The borders, one or both, can also be changed by a separate operating aid, linear or curved. This operating aid is not presented separately but is left up to the technical understanding of the reader.

What all variants have in common is that length 31″ is changed.

In FIG. 6, it has already been enlarged compared to FIG. 5. By zooming in, more configurations appear in selection window 80, whereby selection window 80 is part of the overall display on the screen (display device) as a “functionally linked” function field. It can therefore influence the ability to interact with the user.

Function field 80 can display further configurations after similarity area 31 is expanded. In the example, accessible configuration D is also included in function field 80 under configurations A, B and C.

This intersection point is named z₄ in configuration area 1 and is located on Pareto frontier 501 of configuration D. It cannot currently be reached by second operating aid 22; only three selectors 22a, 22b and 22c are specified here. Nevertheless, function field 80 indicates in advance that a further configuration is available that cannot be achieved due to the limited length of vertical elongated field 31′ (limited size of the interval or limited size of the threshold value).

In this respect, function field 80 is therefore an indication of what could still be, what is brought to the user's attention interactively, but which cannot currently come to fruition due to the interactions in or with the control panel and here in particular due to the (limited) extension of blur interval 31.

Hypothetically, it can be assumed that a further shift of border 31b would also lead to Pareto frontier 501 being reached. However, this could overstrain the similarity. Preferably, the selected area of similarity is below 10% points. It is therefore very limited in order to maintain the similarity in terms of functional significance. It may be recalled that a similarity used interactively here to find alternatives to configurations is technically conditioned, i.e., a technically meaningful similarity is sought that would open up ways out of existing constraints.

It has previously been explained how similarity area 31 is enlarged as a blur (interval) based on FIG. 6. In this enlarged similarity area, however, further options are available to the planner, such as the option that a certain configuration, which is actually shown as being available in the similarity, is not to be selected.

FIG. 7 below explains this.

FIG. 7 shows the system state from FIG. 6 and explains how the representation in FIG. 6 is different. The same similarity area 31 remains, which also has a vertical extension 31″ as shown in FIG. 7. In this similarity area with the aforementioned extension, two operating elements 22a and 22c are located in operating area 2 on operating aid 22. Therefore, no three selectors 22a, 22b and 22c from FIG. 6 are shown here, but selector 22b has been omitted because the user has deactivated or “clicked away” or excluded configuration B in interaction field 82 in function field 80.

Checkbox 82 for configuration B, representative of Pareto frontier 301 and intersection z₂ is not available to the user. The user can select the configurations of available intersection points zᵢ and z₃ from the position of selectors 22a and 22c. For the configuration of the Pareto frontier with point z₃, the user could preferably use Pareto frontier 401 and select the associated configuration, as this Pareto frontier has a lower gradient at intersection point z₃, which can be a criterion for selecting the associated Pareto frontier.

It is also clear from this explanation that all elements of the image display are functionally coupled to one another.

Selecting or deselecting a configuration in function field 80 affects the number and presence of selectors in operating area 2. Changing similarity area 31 has an effect on the vertical extension of blur interval 31′ seen in configuration area 1, and thus on the number of possible and displayed selectors 22a, 22b and 22c, which in turn are shown as selected or deselected or not displayed, depending on the labelling in the checkbox of function field 80.

The checkboxes are to be labelled 81 to 84 as interaction fields; checkboxes 81 and 83 are selected in FIG. 7, corresponding to configurations A and C and consequently to selectors 22a and 22c on operating aid 22 for criterion c2.

Even if only two criteria c1 and c2 are represented in the Figures, more and other criteria can be depicted, as explained in FIG. 3.

The functional link between operating area 2 and configuration area 1 as well as function field 80 has been previously explained and is corroborated here. It is not only possible to operate via selectors 22a or 22c, with which a movement can take place via the functional link on a respective Pareto frontier—in this case with regard to the selected point of, for example, z₂ when selector 22c is moved. This point does not move horizontally but, in conformity with all other criteria, in conformity with criterion c1 here, i.e., on resulting Pareto frontier 801. The Pareto frontier is also visible in all other criteria but is not shown here as well due to the two-dimensionality, although it is easy to conceive of (for an expert).

Operating or changing the position of selector 22c can also be done by changing the position of the point itself with a mouse pointer or, in the case of a touch panel, by active use of a swiping or sliding movement. The functional coupling ensures that the position of associated selector 22c on the operating aid for criterion c2 is also changed.

The change just described affects target point z₃, as it is selected via configuration C and checkbox 83, i.e., it is visible as selector 22c in the operating aid 22. A displacement of the other visible point z₂ is not displayed in the operating area on operating aid 22, as its configuration (that of Pareto frontier 301) is not selected in function field 80. Corresponding checkbox 82 is deselected.

This shows that operating area 2, which is primarily intended for user operation, is only an option. It can also be operated from configuration area 1, with a view of interactively available target points zᵢ shown.

FIG. 8 shows that excluding a configuration can lead to entire areas being blocked. In other words, by excluding a configuration, resulting Pareto frontier 801 can be changed—specifically restricted. If a configuration is excluded, associated Pareto frontier of the configuration cannot be included in determining resulting Pareto frontier 801. If the Pareto frontier of the excluded configuration determines a section of resulting Pareto frontier 801, i.e., the section of resulting Pareto frontier 801 corresponds to a section of the Pareto frontier of the excluded configuration, resulting Pareto frontier 801 changes. This is usually restricted and can therefore include fewer values or points.

These locked fields are shown with 41 and 42 on the two operating aids 21 and 22. They are depicted from operating area 2 in configuration area 1 by extension 42″ on criterion axis c2 and extension 41″ on criterion axis c1.

These two exclusion areas follow the deselection of configuration C via checkbox 83 in function field 80. As taking this configuration into account for user interaction is not intended at this time, corresponding sections “42” and “41” are not available on criteria c1 and c2. The selection options are thus limited, but it is still possible to select blur interval 32 for criterion c2, with value c11 remaining constant on the axis of criterion c1.

This field 32′ corresponds to selection field 31′ in FIG. 7, except that configuration B is selected here via checkbox 82, so that another control element 22b also appears in operating area 2 on operating aid 22, whereas control element 22c is missing due to the deselection of configuration C by deactivating checkbox 83.

In the example shown, the exclusion of the configuration with configuration 501 leads to a restriction of the selection options. Excluding this configuration eliminates the “best options” of criterion c1.

Associated fields 42″ and 41″ can be marked in color on the display device, ideally in a signal color to indicate their unavailability in the configuration area, but also with a signal color in associated exclusion intervals 41 and 42 in operating area 2.

If the other axis c2 is fixed in all examples, the vertical similarity fields described above extend in a horizontal direction. These orientations are disclosed accordingly as well. The description of a direction or orientation is not a limitation.

As explained above, a score can be determined or calculated for the configurations. The score can be shown on display device 10, such as below the associated configuration in field 80. The score can provide a user with further information about the suitability of a configuration.

FIG. 9 shows a network-orientated representation of the control system via a bus 701 and the digital components used that are coupled to it. In the preferred example, bus 701 is a 64-bit bus via which the components communicate with one another. It can be bidirectional.

For the computing capacity and computing function, CPU 700 (or the processor) can be specified, which reads functions 101, 201, etc. displayed on display device 10 from memory 750 and forwards them to display unit I/O 760 via bus 701. Depending on the technical type of display device 10, the data is processed and visualized in display unit 760.

The navigation environment of FIG. 1 is shown. It is operated via the function pointer M and moved and activated by the user via a mouse device, shown here in the example as trackball M′ (selection of a function at a location of pointer M). Trackball M′ is coupled by radio to a receiver 710 (wirelessly linked), which converts its movement signals and forwards them to bus 701. This coupling does not need to be bidirectional. It can be unidirectional.

Mouse device M′ is assigned to display device 10, e.g., as the trackball, which enables the functions of pointing and operating (triggering a function) at the location where mouse pointer M is displayed.

Mouse pointer M is also used to operate both operating aids 21 and 22 for criteria c1 and c2.

An alternative display device 781 (display) is that of a tablet 780, which is given the same representation of FIG. 1, which is shown on touch-sensitive display 781. As explained above, the screen display or tablet display has a configuration area 1 and operating area 2.

Tablet 780 can be coupled to bus 701 via a wireless coupling, e.g., a Wi-Fi coupling (or connection) with a transmitter and receiver 770. The mouse pointer is replaced by a finger (as shown) and controlled by gestures (e.g., swipe, tap, double tap) on display 781 of screen-enabled or touch-sensitive tablet 780.

Specific configurations 100 and 200, as shown in the example in FIG. 2, and other configurations 300 and 400 can each be coupled to bus 701 via input/output device 731, 732, 733 and 734, respectively. Each of these input/output devices is bidirectional, meaning that it can preset parameters from bus 701 to the respective device for its use after settings are made; preferably, each of these devices 100, 200 has sufficient memory of at least 500 GB so that it can store the preset parameters of the subsequent process and set them independently, i.e., autonomously, during the process.

This example can also be controlled so that the parameters are stored in memory 750 for the duration of the process.

Navigation via the Pareto fronts shown on display device 10 can only be completed by the user in order to transfer them later to the associated device(s) via the respective input/output device.

The calculation of CPU 700 results in a graphical definition of a similarity area 31, which was previously described as a blur interval. It is not shown on either display 10 or touch-sensitive display 781 of tablet 780.

1. Procedure for designing or devising a procedural or chemical process with interactive navigation on a display device (10), whereby
(a) at least a first and a second configuration (A, B, . . . Z) are available for selection, and at least a first Pareto frontier (101, . . . ) is formed for the first configuration (A) and at least a second Pareto frontier (201, . . . ) is formed for the second configuration (B);
(b) a resulting Pareto frontier (801) is or is formed on the basis of the first Pareto frontier (101, . . . ) for the first configuration (A) and the second Pareto frontier (201, . . . ) for the second configuration (B), in particular whereby the resulting Pareto frontier (801) is shown or represented on the display device (10);
(c) at least a first operating aid (21) and a second operating aid (22) are shown on the display device (10) in an operating area (2), with the first operating aid (21) representing a first criterion (c1) and the second operating aid (22) representing a second criterion (c2), whereby a position or setting of a selector (21a) of the first operating aid (21) represents a value of the first criterion (c1) and a position or setting of a selector (22a) of the second operating aid (22) represents a value of the second criterion (c2), and whereby the value of the respective criterion (c1, c2) is changed by moving or operating one of the selectors (21a, 22a) of the respective operating aid (21, 22); and
(d) a point (zᵢ) on the resulting Pareto frontier (801) is determined and/or shown on the basis of the position or setting of the selector (21a, 22a).

2. Procedure according to claim 1, whereby the value of the respective criterion (c1, c2) is increased by moving or operating one of the selectors (21a, 22a) in one direction and/or the value of the criterion (c1, c2) is reduced by moving or operating one of the selectors (21a, 22a) in an opposite direction.

3. Procedure according to one of the preceding claims, whereby the position or setting of the other selector (21a, 22a) is changed by moving or operating one of the selectors (21a, 22a), in particular on the basis of the resulting Pareto frontier (801).

4. Procedure according to one of the preceding claims, whereby it is determined whether a deviation or difference between the value for the second criterion (c2) of the first Pareto frontier (101) for the first configuration (A) and the value for the second criterion (c2) of the second Pareto frontier (201) for the second configuration (B) exceeds a threshold value, in particular when the value of the first criterion (c1) is equal.

5. Procedure according to claim 4, whereby the threshold value can be determined or is determined by a user, in particular when a threshold value selection (60) with a selector (60a) is shown on the display device (10), and the threshold value can be definable or is defined by the selector (60a) of the threshold value selection.

6. Procedure according to claim 4 or 5, whereby, when the threshold value is exceeded, at least two selectors (22a, 22b) are shown on the second operating aid (22), whereby the position or setting of one of the selectors (22a, 22b) represents or shows the value of the second criterion (c2) of the first Pareto frontier (101) for the first configuration (A) and the position or setting of the other of the selectors (22a, 22b) represents or shows the value of the second criterion (c2) of the second Pareto frontier (201) for the second configuration (B).

7. Procedure according to one of claims 4 to 6, whereby, when the threshold value is not exceeded, no more than one selector (22a) is displayed on the second operating aid (22), in particular whereby the position or setting of the selector (22a) represents or shows the value of the second criterion (c2) of the resulting Pareto frontier (801).

8. Procedure according to one of the preceding claims, whereby an interval (31), in particular a blur interval, is or will be defined for the value of the first and/or second criterion (c1, c2).

9. Procedure according to claim 8, whereby one or more configurations (A, B) are determined—and, in particular, output or shown or indicated—whose Pareto frontier (101, 201) for the respective configuration (A, B) includes a point which lies within the interval (31, 42) for the first criterion (c1) and relates to a value of the second criterion (c2), which is derived from the value of the first criterion (c1) and the resulting Pareto frontier (801).

10. Procedure according to claim 8 or 9, whereby the interval (31) for the value of the first criterion (c1) is a first interval (31), and a second interval (41), in particular a blurring interval, is or will be determined for the value of the second criterion (c2).

11. Procedure according to claim 10, whereby one or more configurations (A, B) are determined—and, in particular, output or shown or indicated—whose Pareto frontier for the respective configuration (A, B) includes a point which lies within the first interval (31) for the first criterion (c1) and within the second interval (41) for the second criterion (c2).

12. Procedure according to one of claims 8 to 11, whereby the first interval (31) and/or second interval (41) can be or is determined by a user.

13. Procedure according to one of claims 9 to 12, whereby a score is determined for the specific configurations—and, in particular, is output or shown or displayed—whereby the score represents a suitability of the associated configuration.

14. Procedure according to claim 13, whereby the score of the respective configuration (A, B, . . . ) relates to a selected configuration (A, B, . . . ).

15. Procedure according to claim 13 or 14, whereby one or more of the following features are taken into account to determine the score of the respective configuration (A, B, . . . ):
a difference or deviation of points on different Pareto fronts (101, 102, . . . ) of the configurations (A, B, . . . ) relative to one another, in particular points with reference to a mean value of the first and/or second interval (31, 41)
a curve gradient of the Pareto frontier (101, 102, . . . ) within the first and/or second interval (31, 41)
a certainty in the determination of the Pareto frontier (101, 102, . . . ), in particular a certainty in the determination of the values of the Pareto frontier (101, 102, . . . )
a number of setting options of the configuration (A, B, . . . )

16. Procedure according to any one of claims 1 to 15, whereby one or more configurations (A, B, . . . ) are excluded or can be excluded from the procedure, in particular by a user.

17. Procedure according to claim 16, whereby it is determined whether the exclusion of one or more configurations (A, B, . . . ) changes the value range of the resulting Pareto frontier (801), in particular reducing the value range of the resulting Pareto frontier (801).

18. Procedure according to claim 17, whereby a change in the value range of the resulting Pareto frontier (801) is or will be shown or represented on the display device (10), in particular in the operating area (2) of the display device (10).

19. Procedure according to one of the preceding claims, whereby at least three, preferably at least four, more preferably at least five, still more preferably at least six, most preferably at least eight, Pareto fronts (101, 201, . . . ) are formed for the same number of configurations (101, 201, . . . ), and the resulting Pareto frontier (801) is formed on the basis of the Pareto fronts (101, 201, . . . ) of the configurations (A, B, . . . ).

20. Procedure according to one of the preceding claims, whereby the resulting Pareto frontier (801) is a non-dominated set of a union of the Pareto fronts (101, 102, . . . ), in particular of all Pareto fronts (101, 102, . . . ).

21. Procedure according to any one of the preceding claims, whereby different sections of the resulting Pareto frontier (801) correspond to different Pareto fronts (101, 201, . . . ) of configurations (A, B, . . . ).

22. Interactive graphical user interface for the design or devising of a procedural or chemical process, whereby
(a) a resulting Pareto frontier (801) is formed at least on the basis of a first Pareto frontier (101, . . . ) for a first configuration (A) and a second Pareto frontier (201, . . . ) for a second configuration (B);
(b) at least a first interactive operating aid (21) and a second interactive operating aid (22) are displayed on a display device (10) in an operating area (2), whereby the first operating aid (21) represents a first criterion (c1) and the second operating aid (22) represents a second criterion (c2), whereby a position or setting of a selector (21a) of the first operating aid (21) represents a value of the first criterion (c1) and a position or setting of a selector (22a) of the second operating aid (22) represents a value of the second criterion (c2);
(c) whereby the value of the respective criterion (c1, c2) can be changed by a user by moving or operating one of the selectors (21a, 22a) of the respective operating aid (21, 22); and
(d) a point (zᵢ) on the resulting Pareto frontier (801) can be determined and/or shown on the basis of the position or setting of the selector (21a, 22a) defined by the user.

23. Interactive graphical user interface according to claim 22, whereby the display device (10) shows which of the Pareto fronts (101, 201, . . . ) for the respective configuration (A, B, . . . ) determines the section of the resulting Pareto frontier (801) on which the point (zᵢ) lies or is shown.

24. Interactive graphical user interface according to claim 22 or 23, whereby an interactive threshold value selection (60) with a selector (60a) is shown on the display device (10), whereby a value for a threshold can be changed by moving the selector (60a), whereby, when a deviation or difference between a value for the second criterion (c2) of the first Pareto frontier (101) for the first configuration (A) and the value for the second criterion (c2) of the second Pareto frontier (201) for the second configuration (B) exceeds the threshold value, in particular for an equal value of the first criterion (c1), at least two selectors (22a, 22b) are displayed on the second operating aid (22), whereby the position or setting of one of the selectors (22a, 22b) represents or shows the value of the second criterion (c2) of the first Pareto frontier (101) for the first configuration (A) and the position or setting of the other of the selectors (22a, 22b) represents or shows the value of the second criterion (c2) of the second Pareto frontier (201) for the second configuration (B).

25. Interactive graphical user interface according to claim 24, whereby, if the threshold value is not exceeded, no more than one selector (22a) is shown on the second operating aid (22), in particular whereby the position or setting of the selector (22a) represents or shows the value of the second criterion (c2) of the resulting Pareto frontier (801).

26. Interactive graphical user interface according to one of claims 22 to 25, whereby an interval (31) for the value of the first and/or second criterion (c1, c2), in particular a blur interval, is definable at the interactive graphical user interface.

27. Interactive graphical user interface according to claim 26, whereby the interval (31) is shown or represented on the display device (10) in the area of the resulting Pareto frontier (801) and/or in the area of the first Pareto frontier (101, . . . ) for the first configuration (A) and the second Pareto frontier (201, . . . ).

28. Interactive graphical user interface according to claim 26 or 27, whereby the interval (31) is shown or represented on the display device (10) in the area of the first and/or second operating aid (21, 22).

29. Interactive graphical user interface according to any one of claims 22 to 28, whereby a selection of one or more configurations (A, B, . . . ) is shown on the display device (10) and one or more of the configurations (A, B, . . . ) are excludable or includable by a user, whereby the resulting Pareto frontier (801) is based on Pareto fronts for configurations that are included, in particular the resulting Pareto frontier (801), not based on Pareto fronts for configurations that are excluded.

30. Interactive graphical user interface according to claim 29, whereby a change in the value range or a remaining value range of the resulting Pareto frontier (801) is shown on the display device (10) when an exclusion of one or more configurations (A, B, . . . ) changes the value range of the resulting Pareto frontier (801).

31. Interactive graphical user interface according to any one of claims 22 to 30, whereby the resulting Pareto frontier (801) is shown on the display device (10).

32. System with a processing unit (700) configured to perform the procedure according to any one of claims 1 to 21 or to generate the interactive graphical user interface according to any one of claims 22 to 31.

33. Computer program product that includes instructions which, when the program is executed by a computer, cause the computer to perform the procedure according to any one of claims 1 to 21 or to generate the interactive graphical user interface according to any one of claims 22 to 30.

34. Procedure for designing or devising a procedural or chemical process, whereby
(a) a first Pareto frontier for a first configuration and a second Pareto frontier for a second configuration is or will be provided, and
(b) a resulting Pareto frontier is determined or calculated on the basis of the first Pareto frontier and the second Pareto frontier.
